(19) United States
(12) Patent Application Publication
(10) Pub. No.: US 2017/0352283 A1
(43) Pub. Date: Dec. 7, 2017

(54) SELF-ADMINISTERED EVALUATION AND TRAINING METHOD TO IMPROVE MENTAL STATE

(71) Applicant: Cerekinetic, Inc., Edmonds, WA (US)

(72) Inventor: Michael P. H. Lau, Edmonds, WA (US)

(21) Appl. No.: 15/615,242

(22) Filed: Jun. 6, 2017

Related U.S. Application Data
(60) Provisional application No. 62/346,872, filed on Jun. 7, 2016.

(51) Int. Cl. G09B 5/06; A61B 5/16; A61M 21/00; A61B 5/00; A61B 5/0484

(52) U.S. Cl. CPC G09B 5/06; A61B 5/6803; A61B 5/04845; A61M 21/00; A61B 5/04842; A61B 5/165; A61M 2021/0027; A61M 2021/0044

(57) ABSTRACT

A method and system affects the mental state of a user through cognitive or attention training by providing or displaying a first auditory or visual stimulus to a user, manipulating one or more features of the stimulus to provide or display a second auditory or visual image stimulus, and repeating the steps of manipulating features of the auditory or visual image stimulus to provide or display one or more additional auditory or visual image stimuli to the user. An electronic/computing device receives input from the user, such as EEG signal information, indicating the user's mental state which is evaluated to determine a mood of the user. In response to improvement in the user's mood, the user is rewarded with an additional selected auditory or visual image stimulus. Further, based on improvements in the user's mood, the manipulation of features and provision or display of the stimuli may be adjusted.

### BACKGROUND

### Technical Field

The present application relates to a self-administered neuropsychology method and device to evaluate and train a person for the purpose of improving behavior, including but not limited to improving the mental state of the person.

### Description of the Related Art

Depression is one of the most commonly diagnosed mental disorders. A large population survey showed in any 6-month period, 19.5% of the adult U.S. population, or 1 in 5 adults (over age 18) suffers with a diagnosable mental health disorder. In detail, the lifetime prevalence of depressive disorder is 8.3% in the population (Bourdon et al., 1992). Furthermore, the relapse rate—early return of symptoms after positive response to treatment—is high, 25% after 12 weeks (Keller et al., 1982). The future recurrence rate of depression is as high as 30% (Mueller et al., 1999).

Even more alarming, depression is very prevalent amongst young adults. A survey of 20,500 college students reported that 43.2% admitted to feeling depressed, making it difficult to function at least once in the prior 12 months (Miller et al., 2009). The risks and sequelae that clinical depression poses to the young adults, if not diagnosed and treated early and effectively, is high, severe and costly. While routine screening for depression as part of health maintenance has been advocated, it has not been implemented to any extent, especially amongst the younger adult population. As with any disease process, prevention is the best approach.

However, there is no established way to prevent depression in a larger population. There are not enough mental health workers available to carry out any screening or to implement early treatment or preventive measures even for a limited population, such as young adults.

The exact pathophysiologic cause for depression has not been established, but most likely it is caused by multiple factors. Like many mental disorders, depression most likely represents a spectrum of behavioral manifestations. With multiple causal factors, it would be difficult to treat the full spectrum of depression effectively with one single treatment modality.

Pharmacological treatment, such as SSRI antidepressants, is of limited efficacy. In a review, the poor remission rate of 36.8% in the treatment of depression with an SSRI was reported (Timonen et al., 2008). The same review showed greater efficacy combining an antidepressant with cognitive behavioral therapy for clinical depression; and for mild depression, guided self-help and brief cognitive behavioral therapy (CBT) can be used as first line treatments.

To make cognitive behavioral therapy (CBT) more readily available, and more conveniently administered, computerized CBT programs have been developed and used. Basically, these programs are computerized applications to simulate the training performed by a mental health clinician in person. Since many in-person CBT programs are standardized in a “cook book format, it is feasible to transform them into computerized programs. However, these computerized CBT programs need external clinician supervision to be functional. These programs are designed to only train cognitive functions, but by themselves do not have any way to measure mood improvement outcome or to self-adjust the training to optimize the treatment. All the existing mood evaluation methods, such as Beck Depression Inventory, require a healthcare professional to administer and evaluate the assessment. Therefore, these computerized CBT programs cannot be deployed without the guidance of a mental health clinician. The availability of clinicians remains a limiting factor.

Furthermore, mood evaluation assessments, such as Beck Depression Inventory, are all designed to evaluate mood changes over a period of time—weeks and months, and are not suitable for giving immediate assessment and reward right after the training session in order to give incentives for the user to continue further training or to adjust the training level for optimization.

Many “brain training” video games are in the market. While they try to simulate CBT, they suffer similar deficits of lacking immediate outcome measurement and abilities to adjust the training, and therefore remain just “games.”

### BRIEF SUMMARY

The present disclosure provides solutions to the aforementioned limitations in delivering cognitive behavioral training to a larger population to improve the mental state or even treat early mild depression. In various embodiments, the solutions implementing training programs that enhance attention and cognitive processes in the brain to improve mental state and other behaviors in a way that can be self-administered—without the need of a mental health clinician for immediate supervision. In order to accomplish this objective of self-administration, disclosed herein are methods and systems that include measuring, recognizing and rewarding the improved behavior outcome immediately and independently. Furthermore, in various embodiments, the measured outcome in behavioral improvement is fed back into the system, which enables the system to adjust and optimize the training programs, all without requiring the personal presence of a clinician. The methods and systems herein may be utilized by the general population to help to improve mental health without increasing the burden to mental health clinical workers.

BRIEF DESCRIPTION OF THE SEVERAL VIEWS OF THE DRAWINGS

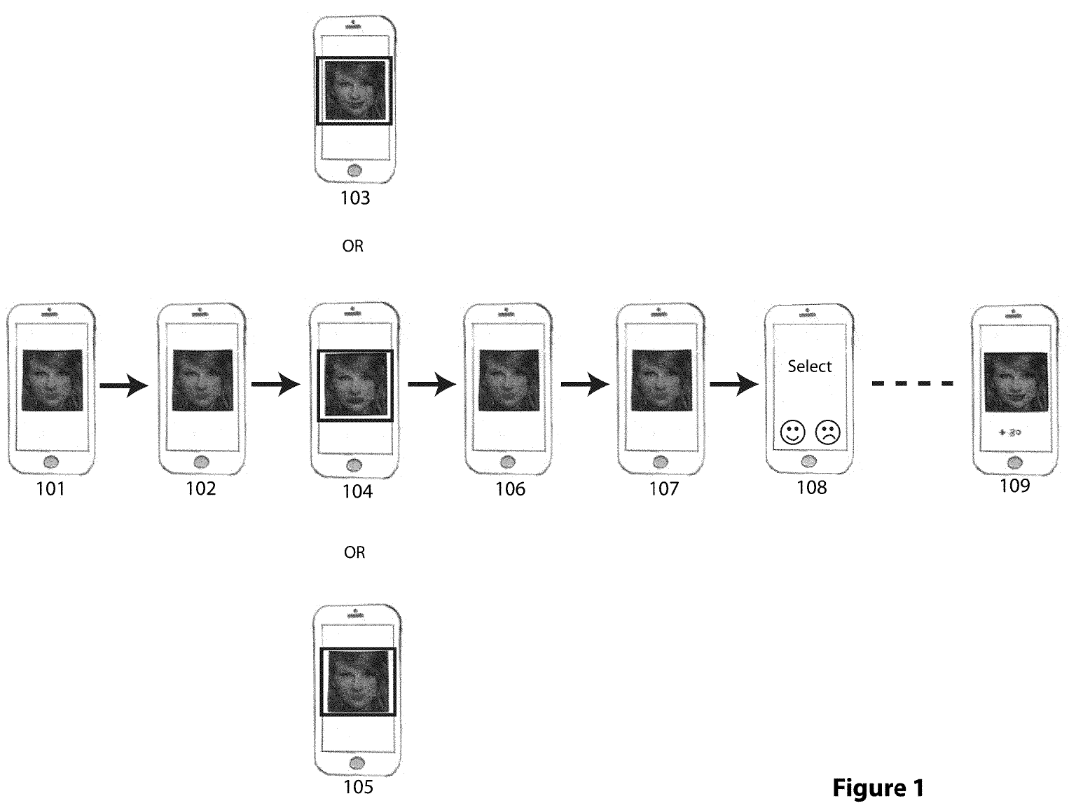

FIG. 1 illustrates an embodiment described herein, in which illusory perception is measured following display of several frames of facial figures, each lasting for a period of time; and

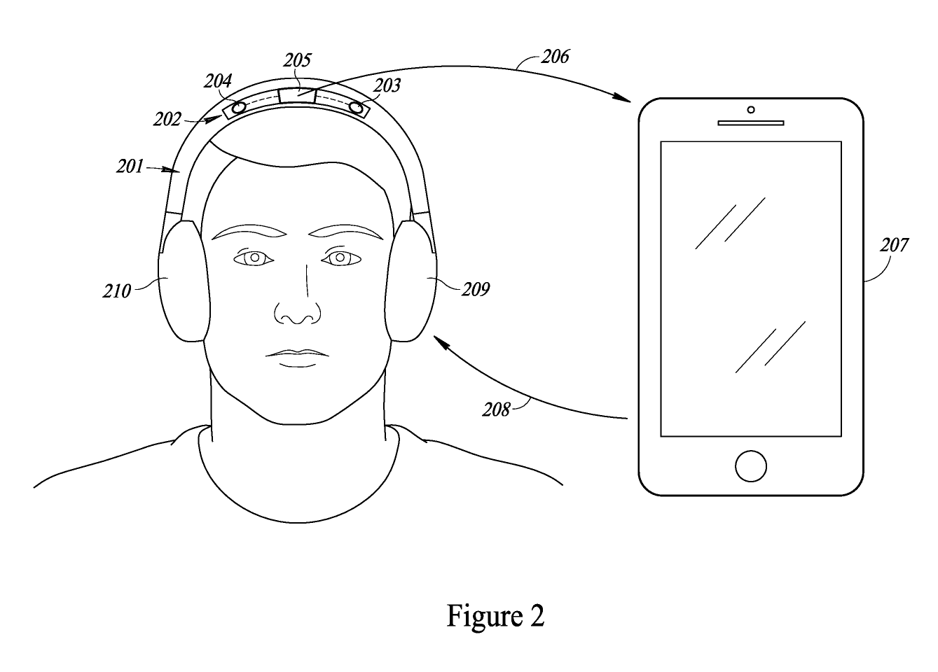

FIG. 2 illustrates an example of a headset that includes an EEG sensor panel with EEG sensors and a microprocessor, including a sensor positioned over the F3 location above the left frontal cortex, and a sensor positioned over the F4 location above the right frontal cortex, for generating neurofeedback signals that are used in training to improve the user's mental state.

### DETAILED DESCRIPTION

Disclosed herein is a method and system to improve a person's mental state using self-administered reward-embedded training in conjunction with an outcome measurement of improved mental state. The method and system also include an option of optimizing the training to further improve mental state through a feedback loop.

Several neuropsychological principles and techniques are utilized, each of which has been shown to be practical through peer reviewed studies, to achieve a novel method and system to improve a person's mental state, and possibly attention, through self-directed training and evaluation. While aspects of the neuropsychological principles and techniques used may be known to those skilled in the art, the unique way to utilize these principles in combination to achieve behavioral improvement as described herein is non-obvious. Furthermore, while this neuropsychology training and evaluation method and system can effectively be deployed through a computerized format, it is not merely an adaptation of a known method to computer applications. Described herein is a novel neuropsychological behavioral improvement tool.

In order to immediately assess the success of, and give incentivizing reward to a user in each training session, at least one preferred aspect of this disclosure is the ability for the user to self-evaluate his or her mental state, such as the user's mood, for example be it happy or sad, before and immediately after a training session. Another preferred aspect of the disclosure is that the measured mental state improvement can provide feedback to the training module to adjust the intensity (e.g., signal strength or tempo) and duration of time of the training to optimize the success in improving the mental state of the user. To further improve the efficacy in behavioral improvement, embodiments of the present disclosure utilize the embedding of reward signals to enhance the efficacy of the training and deliver immediate reward to the user upon improvement of behavior. Furthermore, some embodiments of the disclosure may use auditory stimuli, such as musical rhythms, to entrain the user's cognitive processes and thus enhance the training.

Embodiments of the present disclosure may be implemented by programmed software applications deployed on computing devices including, for example, mobile devices such as an iPhone, iPad, Android-based smart phone or tablets, or computers—either laptop or desktop, or through a cloud-based system accessible via a network such as the Internet.

In accordance with at least one aspect, a user's mental state, for example the user's mood, may be evaluated immediately after each training session based on an understanding that a positive, happy mood tends to increase the probability that the user will visually perceive a happy facial feature, even if no such display is shown—otherwise referred to as an illusory perception. This top-down modulation of visual processing by the user's mood has been documented in a study by Jolij et al. (Jolij, J., et al., 2011). It was demonstrated that listening to happy music improved mood, which tended to increase the probability of the observers reporting seeing a happy face while in fact there was none. The evaluation of mood is performed immediately following a training session, well within the temporary facilitation window of 10 to 15 minutes following musical stimuli, termed as the “Mozart effect” (Rauscher et al., 1995).

Another method to rapidly evaluate a user's mental state (e.g., mood), even in real time, during and after a training session is to use electroencephalography (EEG). There are studies using EEG to evaluate a person's emotion, for example, Ramirez reported the use of EEG measurements to guide musical neurofeedback for treating depression in elderly people (Ramirez, R., 2015). Ramirez measured by EEG the alpha and beta brain waves in the AF3, AF4, F3, F4 locations, and then computed the various ratios of these brain waves in the different locations to determine the arousal and valence of the person. Using neurofeedback, the subjects of the study learned to adjust the loudness of the music to increase arousal. The subjects also adjusted, through neurofeedback, the tempo of the music to increase valence. This study describes a real time assessment of mental state, and using such to perform musical neurofeedback.

The method and system disclosed herein may use reward signals to enhance their effectiveness with users. Reward signals can motivate to enhance perceptual and executive control processes in the user's brain to achieve more efficient goal-directed behavior (Pessoa, L., 2010). Therefore, embedding reward signals in the training processes and using reward signals in the visual and auditory stimuli can improve the training efficacy and motivate the user to strive to achieve the desirable behavior. For example, using a facial character that has known emotional impact to the user as a visual stimulus can function as a reward signal. Seeing one's favorite facial character having a happy face is a strong reward signal. Similarly, playing one's favorite musical pieces is also a strong reward signal.

The method and system disclosed herein may use auditory rhythms to augment the training. Auditory rhythms entrain neuronal oscillations in the brain contributing through a general mechanism by which the brain uses predictive elements in the environment to optimize attention and stimulus perception (Escoffier, N., 2015). Using auditory rhythms, as embedded in training methods or as part of music therapy, can enhance attention and perception training.

In the present disclosure, a feedback mechanism is used to adjust the intensity and duration of time of the training so as to optimize the efficacy of the training to the user. Individuals might have different thresholds in their neural processing and pathways that would respond to a given stimulus, and might respond to different levels of intensity and duration of training as the training progresses. The feedback mechanism fine tunes the optimal training level, be it intensity or duration or both, needed to achieve the desired behavior, hopefully improving with repeated training.

For the training to improve the user's mental state, options in the method for the evaluation of mental state are described in this disclosure. In broad terms, the methods used to evaluate the user's mental state can include at least:

1. Measurement of illusory perception; and

2. Evaluation of mental state by EEG.

Furthermore, multiple training methods, using visual or auditory stimuli, or the combination of both stimuli, are also disclosed. Information from either of the rapid mental state evaluation methods can be used to provide feedback to the training modules to further optimize the training for the improvement of the user's mental state.

### Selection and Processing of Visual Stimuli

A suitable starting point is for a user to choose the visual stimuli for the evaluation and training processes, which can be, for example, a facial figure on which facial expressions would be imposed. The user may choose at least one facial figure provided by the training software, such as a synthetic face generated by composites of facial features or a cartoon of such, or it can be a face of a human model of male or female gender, or a cartoon of such. The facial figure can be only symbolic, in a cartoon form, consisting of just a face, eyes, and mouth, or it can be a photo of a real person. The facial figure can also be of an animal, insect, or even an inanimate object. The user may also download or otherwise supply at least one facial figure of his or her choice comprising the possibilities as described. An objective is to select at least one facial figure as visual stimuli, on which the imposition of a facial expression would be of interest to the user. The selected facial figures can be of a family member, loved ones, pets, or even that of a celebrity. The idea is to select a facial figure that would emotionally generate a maximal reward for the user in perceiving the desirable emotional expression of that facial figure. For example, achieving the perception of a smiling happy face of a celebrity idol or a boyfriend or girlfriend would be gratifying.

The facial figure selected would then be digitized and transformed to pixels of varying granularities of dots in different grey scales or colors, and then be displayed on the screen of the device for the user to accept as recognizable and functional for the purpose as a visual stimulus. The training program would then manipulate the mouth only, for example, or in conjunction with other facial features such as the eyes, eyebrows or cheeks, to create a facial expression of a happy, neutral, or sad mood. Such facial expressions as displayed would then be accepted by the user to be usable as visual stimuli. The transformed facial figure then would be superimposed with digitally created noise patches to obscure the selected facial features which were selected to convey the mood of the facial expression, be it happy, neutral or sad. The purpose of the noise patch is to create an environment of the scrambled facial features through which illusory perception can occur.

### Measurement of Illusory Perception

To assess a user's change of mood right after a training session, and give immediate feedback and reward to the user, a rapid assessment method is desirable. To make the assessment easy and fun, the test is preferably brief, entertaining, and easy to perform. A visual incentive may also be helpful. A technique utilizing illusory perception to assess the mood of the user, as described in a study (Jolij, J., et al., 2011), in a modified form, is used as an example. Other techniques using sensory perception, real or illusory, to assess one's mood can also be used for this aspect of the method.

An interesting visual reward signal, such as the facial figure of a celebrity or a loved one, may be used to show features reflecting moods for the test. The processed facial features of the figure are first displayed without the imposition of noise patch, showing a happy face, and then a sad face. The user can press certain designated keys on a key pad or touch screen to indicate what he or she perceived as the mood of the facial expression, be it happy or sad. For example, the user may press the key “z” for happy, and the key “m” for sad. Alternatively, there the user may press a button corresponding to the mood of the facial expression to be selected by tapping on the touch screen of the device. This step functions as a baseline to acknowledge that the moods are perceivable by the user. A noise patch would then be imposed on the facial features to obscure the critical parts conveying the mood of the facial expression. Several frames, e.g., six frames, of the processed facial figures would then be shown in order to the user on the display, each lasting for a period of time, e.g., approximately 0.1-0.2 seconds or longer. The sequence of the frames may be as follows, as illustrated in FIG. 1:

1. Noise patch imposed (101)

2. Noise patch imposed (102)

3. Mood expression of happy (103), or sad (104), or just a noise patch (105) with no mood expression imposed. There is a frame marker to indicate this frame is one that the user should use to determine the perception of mood of the facial features

4. Noise patch imposed (106)

5. Noise patch imposed (107)

6. Frame for the user to indicate whether he perceived on Frame 3 the mood of happy, sad, or just a random noise patch with no mood imposed (108).

The indication of selection can be as before, for example, by pressing the key “z” for happy, the key “m” for sad, and no selection of keys (or selection of a different key) as an indication of seeing only noise. Specific buttons on the touch screen can also be used for an indication of selection.

The image shown on Frame 3 would be randomly selected by the program to be that of happy, sad, or just a noise patch.

Each sequence of the six frames presented is counted as a trial. 100 to 150 trials can be done in one evaluation setting, for example. In some embodiments, the evaluation setting takes less than four minutes to complete.

The indicated selection of perception of a happy face, sad face, or just noise with no expression of mood seen in each trial would then be compared with the face actually shown in Frame 3.

For a happy or sad face actually shown in Frame 3, the concordance of accurate selection should be high. If not, the duration of Frame 3 can be adjusted to achieve optimal accurate perception. The illusory perception of the mood of the facial feature on Frame 3 is of interest, indicating the dominant tendency of illusory perception for happy or sad face, as influenced by the mood of the user—a happier mood of the user will increase the probability of seeing a happy face on a random noise patch as an illusory perception. The measurement of the illusory perception tendency can be represented in quantitative scale as a “happiness index,” for example, ranging from +50 to −50 corresponding to the percentage of selection of illusory happy expression above or below 50% of the time. Other ways of measurement, such as scoring and scaling, can also be used. For example, if the illusory happy face selection is over 50%, a clearly smiling face of the facial character of choice, such as a celebrity or a loved one can be displayed as a reward (109), maybe even with increasing clarity of the facial features as the index becomes more positive.

The result of the measurement of illusory perception can be used in the feedback loop for optimization of the behavioral training, including mood, anxiety, attention, or addiction training.

### Selection and Processing of Auditory Stimuli

In some embodiments, the users may select their favorite pieces of music to be loaded into a smart phone, mobile device, or other computing device or computer as auditory stimuli. Preferably, the users select their favorite musical pieces that also have faster tempo and of major mode—characteristics of music that are known to improve arousal and mood (Husain, G., 2002). The users load the selected pieces of music into the smart phone, mobile device, or other computing device or computer. A mobile app or computer program already loaded in the smart phone, mobile device, computing device, or computer processes the musical pieces to change the volume and tempo of the music being played. The musical pieces and the modification of the volume and tempo thereof serve as variable auditory stimuli. In addition, a background auditory rhythm of adjustable frequency and volume can be superimposed over the music to serve as additional auditory stimuli, especially for the purpose of auditory entrainment of brain waves. The modification of the volume and tempo of the music, and the frequency and volume of the background rhythm can be modified by direct manual input by the user through the smart phone, mobile device, computing device, or computer, or indirectly through neurofeedback from the user.

### Evaluation of Mental State by EEG

Electroencephalography (EEG) has been used to evaluate a person's mental state, including arousal, mood and attention. EEG captures the brain waves generated from the electric activities of the neurons in the brain through surface sensors placed at the various locations near the skin surface of the head. The brain generates electric waves of different frequencies of varying amplitudes from the various locations within the brain. By analyzing the amplitude of the various wave bands, in conjunction with defining the locations in the brain that are generating those specific brain waves, one can gain understanding of and evaluate the neural activities at the various part of the brain, and furthermore deduce the mental state associated with those neural activities. There are several studies reporting the detection of emotional states using the EEG (Ramirez 2012). Depending on the needs of evaluation of certain specific mental state(s), specific EEG studies can be directed to meet those needs.

EEG sensors, ideally noncontact in type, but can be also contact type with or without conductive gel or fluid, are built into, or attached to a headset. Such headset can be a flexible headband, or a hat or a headset with multiple arms. There should be at least one EEG sensor built into a sensor panel. The sensor panel or panels are attached to, or built into the headset. The EEG sensors can be flexible or rigid. The sensors are connected to microprocessors where the EEG brain waves will undergo signal processing and information processing. The resultant EEG signals and data can then be transmitted wirelessly or through a cable to the smart phone, mobile device or computer.

One embodiment of using EEG to evaluate mood, for example, involves the capturing the EEG brain wave signals from the AF3, F3, AF4, and F4 locations on the head by the sensor panels attached to the headset over those locations. The sensors and microprocessors may be programmed to identify, by filtering, the alpha (8-12 Hz) and the beta (12-28 Hz) brainwaves from the four locations mentioned over the prefrontal cortex. The EEG data processing can be performed by the method, e.g., in a manner adapted from Ramirez (Ramirez 2012). In such processing, the ratio of the beta waves and the alpha waves from the four locations is computed to determine the arousal level of the user. Beta waves are usually associated with alert or excited state of mind, whereas alpha waves are usually associated with a relaxed state. Therefore, the beta/alpha ratio could be used to evaluate the arousal state of the user. To evaluate the emotional state-affective valence—of the user, comparison of the activation levels of the two cortical hemispheres is used. Inactivation of the left frontal cortex is an indication of withdrawal response and negative emotion, whereas inactivation of the right frontal cortex is an indication of approach response and positive emotion. Therefore, by comparing the activities of the alpha waves —indication of relaxation and inactivation, and the beta waves—indication of excitation and activation, between the left and right frontal cortex (at the F3 and F4 location, correspondingly), the affective valence of the user can be evaluated.

### Training Optimization Feedback Loop

A purpose of the behavioral training is to optimize the neurobehavioral pathways in the user's brain, such that the user can apply the training with decreasing effort required, or even in automation, to achieve a desired behavioral state. This behavioral state improvement can be applied to mood, attention, anxiety, compulsion, or addictive disorders. For example, an increasing tendency of perceiving a happy smiling face of a celebrity idol or boyfriend or girlfriend would help to improve the depressive mood of the user. Similarly, a happy face perception would convey approval of a loved one, such as parents or spouse, in improving compulsive or addictive behavioral problems.

For example, if the measurement of result in the illusory perception evaluation showed a better efficacy of computerized attention control training versus that of music therapy, the former training method would be selected as a primary training method. Another example would be in the selection of different pieces of music in music therapy. Certain music might have better efficacy in improving the user's mood as measured by the illusory perception trials. The more effective pieces of music will be selected for the training program, and as the user's mood continues to improve, the feedback signal from the illusory perception measurements can be used to shorten the piece of music used as therapy. This could even progress to the point that a few notes of the music piece, either through the device or even through memory of the user, can generate the desirable behavioral effect, be it improvement of mood or avoiding addictive or compulsory behavior, such as those relating to chronic physical or emotional pain.

For embodiments using EEG to evaluate the user's mental (or emotional) state and arousal, the processed EEG data can be used for neurofeedback to adjust music and rhythm training with an aim to improve mood or other mental state. As described, the beta/alpha waves ratio reflects the arousal state of the user. By using the beta/alpha ratio for neurofeedback, the user can train to increase or decrease the volume and/or tempo of the music played to adjust the user's arousal level. Similarly, neurofeedback can be used to improve mood. Inactivation of the left frontal cortex is an indication of withdrawal response and negative emotion. Since alpha waves indicate inactivation, using neurofeedback training to decrease the alpha wave activities at the left frontal cortex—i.e., decreasing the inactivation—by increasing the tempo of the music, the affective valence should improve (Ramirez, 2015). Similar logic can be applied to the measurement and processing of other brain waves, for example beta waves, for neurofeedback training, or working with the EEG data from the right frontal cortex, or other locations of the brain to do such.

### Training Modules

At least one training module can be deployed to train a user to improve his mood, alleviate anxiety, or suppress addictive compulsions. The training modules may comprise, but are not limited to, the following examples:

1. Music therapy and rhythm training.

2. Cognitive control training.

3. Attention training.

4. Working memory training.

5. Any cognitive exercise training.

6. Any of the above training augmented by administration of pharmacologic therapies.

7. Any of the above training augmented by administration of deep brain stimulation therapies.

The auditory and/or visual image stimulus for the purpose of training as described above may include a piece of music. In some embodiments, the piece of music may be associated with a visual image. Alternatively or in addition, the auditory and/or visual image stimulus for the purpose of training as described may include auditory tones comprising chanting, meditation mantra, acoustic rhythms, or any natural or synthetic noise.

The auditory and/or visual image stimulus for the purpose of training as described above may include segments of natural or synthetic visual images generated by photography, video, or computer simulation.

The auditory and/or visual image stimulus for the purpose of training as described above may include a synthesis or combination of both the auditory and visual image stimuli, in a two dimensional display, a three dimensional display, or a virtual reality format.

While the training modules may be deployed separately from the computing device, preferred embodiments of the present disclosure include programming the training modules into the device itself for deployment. Furthermore, with incorporation of the training programs in a computerized format in the device, the measurement of, the reward for, and the ability to build in a feedback loop system with the successful behavioral outcome tends to be more feasible.

An example of a training module under the category of attention training could take the form of a modified version of the Attention Training Technique (ATT) as described by Wells (Wells, 2009). Rhythms are built in the auditory stimuli specifically to optimize attention and stimulus perception in the brain to enhance the attention and perception training (Escoffier, N., 2015).

A software program is built that can be deployed (i.e., executed by a processor or other processing circuitry) in a—computing device, such as a laptop or desktop computer, a mobile device including, for example, a smart phone or tablet, or through the Internet using one or more available computing resources. In operation, the program may display a facial character of the user's choice—preferably one that has maximal emotional impact on the user—on the display screen. The user would focus on that facial character, who may be programmed to give verbal instructions to the user over a stereo headphone or speaker system. The facial character may first ask the user to focus only on the character's voice (S1), ignoring all other background sounds, created in the device or otherwise. Then a command would be given for the user to focus only on another sound (S2), such as a rhythmic drum beat, with full attention. The user is then asked to focus on a third sound (S3), such as a rhythmic repeating piano chord. Then a command may be given to listen to a fourth target sound (S4) with a spatial element, such as a rhythmic sound of a flute coming out of the left side of the stereo system—headphone or speaker. The instructions may then be repeated for additional sounds (S5-S7), with different sound character and spatial orientation of the sound. This portion of training may be repeated and go on for about 5 minutes, for example.

The user is asked to focus on only one sound, then quickly shift full attention to another sound, then yet another sound, with different character and spatial source, and so on. For example, in sequence such as S1 to S4, to S3, to S7 . . . This is to train for quick attention shifting. This part of training can go on for 5 minutes, for example.

The last part of the training exercise would be to expand the user's attention to absorb all the sounds and from all the spatial source locations at the same time. The user will try to identify the number of sounds that he or she can hear simultaneously. This part of training would last for two minutes, for example. Then, this training module is concluded.

The illusory perception measurements as described earlier herein can be applied before and after execution of the training module to evaluate mood change of the user as the result of the training.

Another embodiment of training, for example, is music therapy. It has been shown in multiple studies that certain music can enhance mood and cognitive functions. Husain reported that musical tempo (fast or slow) manipulations affected arousal but not mood, whereas mode (major and minor) manipulations affected mood but not arousal (Husain, G. et al., 2002). Even listening to the musical stimuli for only 10 minutes produced measurable cognitive and mood effect. In this embodiment, selected pieces of happy-sounding music, in major mode and fast tempo, are used for training. The rhythm component of the music can be amplified to further augment stimulus processing as to enhance attention optimization and stimulus perception (Escoffier, et al., 2015). Additional background auditory rhythm stimuli such as, for example, snare and bass drum sounds at 1.3 Hz frequency as described in a study (Escoffier, 2015), can also be used to entrain the brain waves and the associated brain activities.

Furthermore, this embodiment can leverage embedded reward signals to enhance the training (Pessoa, et al., 2010). For example, the user can select his favorite music idol as his facial figure visual stimuli for mood assessment, and then also select the happy-sounding music pieces by the same music idol as the auditory stimuli for training. The cross modalities of stimuli—visual and auditory—enhanced by the embedding of reward signals could optimize the mood and cognition improvement.

With 3-dimensional technology and virtual reality technology, the visual and auditory stimuli can be further enhanced to augment the training. This can be applied, for example, to the Attention Training technique (ATT), as disclosed above, where the auditory stimuli, even in conjunction with visual stimuli, can be in a 3-D format or in a virtual reality format. These formats can also be adapted to the deployment of music therapy as disclosed, especially with the combination of visual, auditory stimuli, and reward signals as described.

Another embodiment of training, for example, is the Pace Auditory Additional Task PASAT (Gronwall, 1977), or a modified form of such (Siegle, 2007)—termed as Cognitive Control Training. The examples of Attention Training Technique and Cognitive Control Training are versions of cognitive exercise training, with the objective to improve mood and other behaviors. The PASAT involves asking the participants to add each new digit to the digit that preceded it. The speed of presenting the digits will then speed up as the training progresses to add difficulties in order to work on executive control in addition to working memory. Working memory is considered to be one of the most important mental faculties critical to cognitive abilities such as planning, problem solving and reasoning. There are many working memory training programs, some in a computerized format, available for cognitive training.

An embodiment of training utilizes musical rhythm to enhance a technique modified from the Pace Auditory Additional Task training to improve mood and behaviors. The user will tap keys to mimic the rhythm and number of beats presented, and add each new set of beats with rhythm to the set preceding it. The speed and complexities of the beats and rhythm sets will increase as the training progresses to enhance working memory and executive control. Reward signals, such as favorite music pieces, can be incorporate into the auditory stimuli for training. Similarly, images of idols or loved ones can be incorporate as visual stimuli in the training.

Pharmacologic therapies are often used in conjunction with cognitive therapies to enhance the efficacy in treating behavioral disorders. For depression, drugs classified as antidepressants are used. There are several classes of antidepressants, with differing mechanisms in their therapeutic action. For example, the most common class of antidepressants is the Selective Serotonin Reuptake Inhibitors SSRIs, with brand names such as Prozac, or Lexapro, for example. Some of the other classes, for example, are tricyclic antidepressants, and monoamine oxidase inhibitors. For anxiety, beta-blockers, such as propranolol, can be used to decrease the physiologic response of anxiety to facilitate cognitive training. The adjunctive use of medications and cognitive training is another embodiment being disclosed.

Deep brain stimulation using electric currents, magnetic stimulation or ultrasound have been used by themselves, or to augment pharmacologic therapies, in treating behavioral disorders. Another embodiment being disclosed is to used deep brain stimulation techniques in conjunction with the cognitive training to enhance the therapeutic efficacy in improving mood and other behaviors.

### Self-Administered Evaluation and Training Device to Improve Mental State

An embodiment comprising a mood evaluation, training module, and feedback loop to improve the user's mental state, as an example, is now described. The evaluation method, the training module, or the way to practice the feedback loop can be any of, or a combination of, those ways described previously herein. In this disclosed embodiment, non-contact EEG sensors are attached to a headset. The EEG sensors can be built in the headset individually, or the sensors can be embedded in a sensor panel, with specific configurations, which is then attached to a headset, in a fixed or detachable manner. More than one sensor panel can be used. The headset can be a headband, a hat, a headset with complex configurations for specific EEG reception design, a headset with ear phone of special design, or a commercially available earphone headset. The EEG sensors and/or the sensor panels can be flexible or rigid. The EEG sensors are connected to microprocessors to have the EEG signals and data processed and transmitted, preferably wirelessly. The transmitted EEG signals and data can be received by a smart phone, mobile device, computing device, or computer.

The EEG signals and/or processed data can then be further processed in the smart phone, for example, to generate commands to adjust the music and rhythm training module—with adjustable volume, tempo or background rhythm control—in play through the smart phone. For example, the user is playing a favorite piece of music through the music and rhythm module in the smart phone. As part of training to improve the user's mental state, the user will attempt to use neurofeedback to increase the tempo of the music. The underlying neuropsychological principle is that inactivation of the left frontal cortex is an indication of withdrawal response and negative emotion. The objective of the training to improve the mood of the user is to decrease the inactivation of the left frontal cortex—a major activity of the brain is the inactivation of certain parts of the brain. Since alpha waves indicate inactivation, by neurofeedback training using the EEG data input from the sensors on the headset, the user is training to decrease the alpha wave activities at the left frontal cortex—decreasing the inactivation—by using neurofeedback to increase the tempo of the music, and thereby improve the affective valence. The music and rhythm training module in the smart phone, for example, would evaluate the alpha waves from the F3 location over the left frontal cortex. The user is trained to direct the user's emotional state to a more positive plane, and through neurofeedback, to decrease the alpha waves amplitude by adjusting the tempo of the music upward through the training module neurofeedback mechanism. The feedback loop would thus continue to improve the training process. The neurofeedback training, for example, can also include using EEG alpha waves signals from F4, over the right frontal cortex. In this case, the feedback training is to increase the alpha wave amplitude of F4, implying inactivation of the negative emotion associated with the increasing right frontal cortex activities.

It is also possible to use the disclosed method and system to feed in a background rhythm, with or without music, to specific unilateral earphones as to entrain the brainwave activities of the corresponding cerebral hemisphere to enhance the training and the response to the neurofeedback for improvement of the user's mental state.

The various combinations of EEG wave data from the various locations on the head may be analyzed and used for neurofeedback through the different training modules, utilizing the non-contact EEG sensors in the headset in various specific configurations, to improve the mental state of the user, including affective valence, arousal and attentions, for example.

Another embodiment of training, for example, such as the Pace Auditory Additional Task PASAT (Gronwall, 1977), or a modified form of such (Siegle, 2007)—termed as Cognitive Control Training described previously, can also be deployed through the above-disclosed EEG data driven evaluation and neurofeedback mechanism to improve the user's mental state.

FIG. 2 illustrates an example of the embodiment disclosed above. The headset comprises a headband (201) and an EEG sensor panel (202) which is fixed to, or temporarily attached to, the headband (201). Non-contact EEG sensors (203 and 204) and a microprocessor (205), with or without power supply, are attached to or embedded within the EEG sensor panel (202). In this illustration, the sensor 203 is over the F3 location above the left frontal cortex, and the sensor 204 is over the F4 location above the right frontal cortex. EEG signals captured by the sensors 203 and 204 are transmitted to the microprocessor (205) to be processed. The processed EEG information (i.e., signals and/or data) are then transmitted (206), preferably wirelessly, to a smart phone (207) where a training module, for example a music and rhythm training module as discussed earlier herein, will process the EEG information to generate neurofeedback signals to adjust the tempo and volume of the training music and/or background rhythm in a feedback loop. The music and rhythm stimuli are transmitted wirelessly to earphones (209 and 210) situated on the user's ears. Through this setup, the user uses neurofeedback to train to improve the user's mental state.

The user can thus implement methods of training to improve his or her mental state using various cognitive or attention training processes. In at least some embodiments, the improvement of mental state increases the probability of illusory perception, e.g., of seeing a happy face, thus rewarding the person with increasing illusory perception tendency of seeing an electronically generated facial expression of improved mental state. The increase in frequency of such positive illusory perception can be a measure of success of the training. Additionally, the improvement in mental state is quantified and feedback is provided to the training modules, providing the training processes an opportunity to adjust the intensity and/or duration of the training to optimize the training to achieve the desired mental state improvement.

The various embodiments described above can be combined to provide further embodiments. All of the publications referred to in this specification are incorporated herein by reference, in their entirety. Aspects of the embodiments can be modified, if necessary to employ concepts of the various publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

I claim:

1. A method to affect the mental state of a user through cognitive training using visual image stimuli, the method comprising:
generating, by electronic processing, a first facial image of a human, animal, or cartoon character;
displaying, on an electronic device, the first facial image to a user;
manipulating facial features of the first facial image to produce a second facial image representing a mood of the face, including at least one of happy facial features, neutral facial features, sad facial features, or absence of such happy, neutral, or sad facial features;
displaying, on the electronic device, the second facial image to the user;
repeating the steps of manipulating facial features to produce one or more additional facial images representing a mood of the face and displaying, on the electronic device, the one or more additional facial images to the user;
receiving input from the user indicating a particular mood that the user perceives a particular displayed facial image to be displaying, including a happy mood or a sad mood;
comparing the user input of perceived mood of the particular displayed facial image to a predetermined actual mood of the particular displayed facial image to determine an accuracy of the user's perception of the actual mood;
rewarding the user with additional selected visual stimuli in response to improved accuracy of the user's perception of the actual mood; and
based on the determined accuracy of the user's perception of actual mood, adjusting at least one of intensity or duration of time in which selected facial features of facial images are manipulated and displayed on the electronic device.

2. The method of claim 1 further comprising providing auditory stimuli that include auditory tones comprising music, chanting, meditation mantra, acoustic rhythms, or any natural or synthetic noise.

3. The method of claim 1 wherein the visual image stimuli include segments of natural or synthetic visual images generated by photography, video, or computer simulation.

4. The method of claim 1 wherein the visual image is combined with auditory stimuli, and displayed, on the computing device, in a two dimensional display, a three dimensional display, or a virtual reality format.

5. A method to affect the mental state of a user through cognitive or attention training using auditory and/or visual image stimuli, the method comprising:
generating, by electronic processing, a first auditory or visual stimulus;
providing or displaying, on an electronic device, the first auditory or visual image stimulus to a user;
manipulating one or more features of the first auditory or visual image stimulus to produce a second auditory or visual image stimulus;
providing or displaying, on the electronic device, the second auditory or visual image stimulus to the user;
repeating the steps of manipulating features of the auditory or visual image stimulus to produce one or more additional auditory or visual image stimuli, and providing or displaying, on the electronic device, the one or more additional auditory or visual image stimuli to the user;
receiving, by the electronic device, input from the user indicating the user's mental state in reaction to the provided or displayed auditory or visual image stimuli, wherein the input from the user includes electroencephalography (EEG) signal information collected from one or more sensors placed at one or more locations near a surface of the user's head;
evaluating the input from the user to evaluate the user's mental state to determine a mood of the user;
rewarding the user with an additional selected auditory or visual image stimulus in response to a determined improvement in the mood of the user; and
based on the determined improvement in the mood of the user, adjusting the manipulation of the features of the auditory or visual image stimuli that are provided or displayed on the electronic device.

6. The method of claim 5 wherein the auditory stimuli include music and rhythm therapy.

7. The method of claim 5 further comprising administering one or more pharmacologic therapies to the user in coordination with providing or displaying the auditory or visual image stimuli to the user.

8. The method of claim 5 further comprising administering one or more deep brain stimulation therapies to the user in coordination with providing or displaying the auditory or visual image stimuli to the user.

9. The method of claim 5, wherein the auditory or visual image stimulus includes auditory tones comprising music, chanting, meditation mantra, acoustic rhythms, or any natural or synthetic noise.

10. The method of claim 5, wherein the auditory or visual image stimulus includes segments of natural or synthetic visual images generated by photography, video, or computer simulation.

11. The method of claim 5, wherein the auditory or visual image stimulus include a combination of both auditory and visual image stimuli that is provided and displayed in a two dimensional display, a three dimensional display, or a virtual reality format.

12. A system configured to affect the mental state of a user through cognitive or attention training using auditory and/or visual image stimuli, the system comprising:
a computing device that includes one or more processors; and
a headset wearable by a user to collect electroencephalography (EEG) signal information of the user, wherein the headset is configured to place one or more sensors at one or more locations near a surface of the user's head, and wherein the headset transmits the EEG signal information to the computing device,
wherein, in response to execution of computer-executable instructions, the one or more processors of the computing device operate to:
generate a first auditory or visual stimulus;
provide or display, via the computing device, the first auditory or visual image stimulus to the user;
manipulate one or more features of the first auditory or visual image stimulus to produce a second auditory or visual image stimulus;
provide or display, via the computing device, the second auditory or visual image stimulus to the user;
repeat the operations of manipulating one or more features of the auditory or visual image stimulus to produce one or more additional auditory or visual image stimuli, and provide or display, via the computing device, the one or more additional auditory or visual image stimuli to the user;
receive, by the computing device, input from the user indicating the user's mental state in reaction to the provided or displayed auditory or visual image stimuli, wherein the input from the user includes EEG signal information collected by the one or more sensors placed near the surface of the user's head;
evaluate the input from the user to evaluate the user's mental state to determine a mood of the user;
reward the user with an additional selected auditory or visual image stimulus in response to a determined improvement in the mood of the user; and
based on the determined improvement in the mood of the user, adjust the manipulation of one or more features of the auditory or visual image stimuli that are provided or displayed via the computing device.

13. The system of claim 12, wherein the computing device is connected to a cloud computing system to receive commands in relation to providing or displaying the auditory or visual image stimuli.

14. The system of claim 12, wherein the computing device is a mobile device coupled to the headset via a communication network.

15. The system of claim 12, wherein the auditory stimulus provided by the computing device includes music and rhythm therapy.

16. The system of claim 12, further comprising administering one or more pharmacologic therapies to the user in coordination with providing or displaying the auditory or visual image stimulus to the user.

17. The system of claim 12, further comprising administering one or more deep brain stimulation therapies to the user in coordination with providing or displaying the auditory or visual image stimulus to the user.

18. The system of claim 12, wherein the auditory or visual image stimulus includes auditory tones comprising music, chanting, meditation mantra, acoustic rhythms, or any natural or synthetic noise.

19. The system of claim 12, wherein the auditory or visual image stimulus includes segments of natural or synthetic visual images generated by photography, video, or computer simulation.

20. The system of claim 12, wherein the auditory or visual image stimulus includes a combination of both auditory and visual image stimuli that is provided and displayed in a two dimensional display, a three dimensional display, or a virtual reality format.
